SEQUENCE LISTING (10 sequences)

SEQ ID NO: 1 - protein, 15 aa, Mus musculus
ASHPTILYTLLSPGP

SEQ ID NO: 2 - protein, 25 aa, Mus musculus
QEPVSPKKKENALLRYLLDKDDTKD

SEQ ID NO: 3 - protein, 24 aa, Mus musculus
SPSSHSSLTERHKILHRLLQEGSP

SEQ ID NO: 4 - protein, 24 aa, Mus musculus
QKPTSGPQTPQAQQKSLLQQLLTE

SEQ ID NO: 5 - protein, 20 aa, Mus musculus
PCQGSASHPTILYTLLSPGP

SEQ ID NO: 6 - protein, 20 aa, Mus musculus
VAEAPVPSILKKILLEEPNS

SEQ ID NO: 7 - protein, 24 aa, Mus musculus
GHGEDFSKVSQNPILTSLLQITGN

SEQ ID NO: 8 - protein, 24 aa, Mus musculus
SGNLVPDAASKHKQLSELLRGGSG

SEQ ID NO: 9 - protein, 25 aa, Mus musculus
GHSFADPASNLGLEDIIRKALMGSF

SEQ ID NO: 10 - protein, 27 aa, Mus musculus
QAVQEHASASTNMGLEAIIRKALMGKY

(19) United States
(12) Patent Application Publication
(10) Pub. No.: US 2007/0026059 A1
(43) Pub. Date: Feb. 1, 2007

(54) Ligands for orphan nuclear hormone receptor steroidogenic factor-1 (SF-1)

(76) Inventors: Huaqiang Eric Xu, Grand Rapids, MI (US); Yong Li, Grand Rapids, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/348,207

(22) Filed: Feb. 6, 2006

Related U.S. Application Data
(60) Provisional application No. 60/649,562, filed on Feb. 4, 2005.

(51) Int. Cl. A61K 31/685; G01N 33/53; A61K 9/127

(52) U.S. Cl. 424/450; 435/7.1; 514/78

(57) ABSTRACT

The 1.5 Å crystal structure of the orphan nuclear receptor steroidogenic factor 1 (SF-1) ligand binding domain in complex with an LxxLL motif from a co-regulator protein is disclosed. The structure reveals the presence of a phospholipid ligand in a surprisingly large pocket (˜1600 Å³), with the receptor adopting the canonical active conformation. The bound phospholipid is readily exchanged and modulates SF-1 interactions with co-activators. Directed mutations that alter the ligand binding pocket disrupt SF-1/co-activator interactions and reduce SF-1 transcriptional activity. Also disclosed is a method to screen chemical libraries for compounds with high specificity or binding affinity for SF-1, thereby permitting the discovery of agonist or antagonist drugs useful in treating dyslipidemias and/or in promoting cholesterol homeostasis.

### CROSS REFERENCE TO RELATED APPLICATIONS

This application claims benefit of provisional application Ser. No. 60/649,562, filed Feb. 4, 2005, entitled LIGANDS FOR ORPHAN NUCLEAR HORMONE RECEPTOR STEROIDOGENIC FACTOR-1 (SF-1), the entire contents of which are incorporated herein in their entirety.

### FIELD OF THE INVENTION

The invention in the field of biochemistry and medicine relates to the discovery of phospholipid ligands for the orphan nuclear receptor steroidogenic factor 1 (SF-1) which was considered before as being ligand independent and constitutively active. The bound phospholipid is readily exchanged and modulates SF-1 interactions with co-activators. The invention provides an approach to screening for and/or designing synthetic SF-1 ligands with better pharmacokinetic properties than phospholipids, and which are drug candidates for treating SF-1 related diseases such as dyslipidemias, endocrine disorders, and/or improving cholesterol homeostasis.

### BACKGROUND OF THE INVENTION

Steroidogenic factor 1 (SF-1) is a member of the nuclear receptor family that plays multiple roles in development and metabolism. In mammals, SF-1 is required for differentiation of endocrine glands and sexual development (Parker, K. L., Rice, D. A., Lala, D. S., Ikeda, Y., Luo, X., Wong, M., Bakke, M., Zhao, L., Frigeri, C., Hanley, N. A., et al. (2002). Steroidogenic factor 1: an essential mediator of endocrine development. Recent Prog Horm Res 57, 19-36; Sadovsky, Y., and Dorn, C. (2000). Function of steroidogenic factor 1 during development and differentiation of the reproductive system. Rev Reprod 5, 136-142). Mice devoid of SF-1 lack adrenals, gonads, and the ventromedial hypothalamic nucleus and have impaired expression of pituitary gonadotrope markers (Ingraham, H. A., Lala, D. S., Ikeda, Y., Luo, X., Shen, W. H., Nachtigal, M. W., Abbud, R., Nilson, J. H., and Parker, K. L. (1994). The nuclear receptor steroidogenic factor 1 acts at multiple levels of the reproductive axis. Genes Dev 8, 2302-2312; Luo, X., Ikeda, Y., and Parker, K. L. (1994). A cell-specific nuclear receptor is essential for adrenal and gonadal development and sexual differentiation. Cell 77, 481-490). In adults, SF-1 controls the synthesis of sex steroids, glucocorticoids and steroidogenic Factor-1s by regulating multiple steroidogenic enzymes (Bakke, M., Zhao, L., Hanley, N. A., and Parker, K. L. (2001). SF-1: a critical mediator of steroidogenesis. Mol Cell Endocrinol 171, 5-7). SF-1 thus serves as a master regulator of endocrine development and function.

SF-1 is part of a nuclear receptor subfamily that includes the Drosophila protein FTZ-F1 and the vertebrate protein LRH-1. Members of this subfamily play key roles during development and in adult homeostasis by binding as monomers to DNA sequence elements in the regulatory regions of target genes (Shen, W. H., Moore, C. C., Ikeda, Y., Parker, K. L., and Ingraham, H. A. (1994). Nuclear receptor steroidogenic factor 1 regulates the mullerian inhibiting substance gene: a link to the sex determination cascade. Cell 77, 651-661; Wilson, T. E., Fahrner, T. J., and Milbrandt, J. (1993). The orphan receptors NGFI-B and steroidogenic factor 1 establish monomer binding as a third paradigm of nuclear receptor-DNA interaction. Mol Cell Biol 13, 5794-5804).

Notably, SF-1 and all of its closely-related family members remain “orphans” in that it is not known whether their transcriptional activity is regulated by physiologic ligands. In cell-based reporter assays, SF1 appears to be constitutively active since it stimulates transcription in the absence of any exogenous ligand. One report claimed that SF-1 was activated by oxysterol metabolites of cholesterol (Lala, D. S., Syka, P. M., Lazarchik, S. B., Mangelsdorf, D. J., Parker, K. L., and Heyman, R. A. (1997). Activation of the orphan nuclear receptor steroidogenic factor 1 by oxysterols. Proc Natl Acad Sci USA 94, 4895-4900); however, others failed to confirm this effect (Desclozeaux, M., Krylova, I. N., Horn, F., Fletterick, R. J., and Ingraham, H. A. (2002). Phosphorylation and intramolecular stabilization of the ligand binding domain in the nuclear receptor steroidogenic factor 1. Mol Cell Biol 22, 7193-7203; Mellon, S. H., and Bair, S. R. (1998). 25-Hydroxycholesterol is not a ligand for the orphan nuclear receptor steroidogenic factor-1 (SF-1). Endocrinology 139, 3026-3029). Whereas it has remained unknown whether SF-1 is regulated by small molecule ligands, its transcriptional activity can be regulated by tissue-specific repressor proteins, including the orphan receptor Dax-1 (I to, M., Yu, R., and Jameson, J. L. (1997). DAX-1 inhibits SF-1-mediated transactivation via a carboxy-terminal domain that is deleted in adrenal hypoplasia congenita. Mol Cell Biol 17, 1476-1483; Nachtigal, M. W., Hirokawa, Y., Enyeart-VanHouten, D. L., Flanagan, J. N., Hammer, G. D., and Ingraham, H. A. (1998). Wilms' tumor 1 and Dax-1 modulate the orphan nuclear receptor SF-1 in sex-specific gene expression. Cell 93, 445-454), and by phosphorylation in the hinge region (Desclozeaux et al., 2002; Hammer, G. D., Krylova, I., Zhang, Y., Darimont, B. D., Simpson, K., Weigel, N. L., and Ingraham, H. A. (1999). Phosphorylation of the nuclear receptor SF-1 modulates cofactor recruitment: integration of hormone signaling in reproduction and stress. Mol Cell 3, 521-526).

Like other nuclear receptors, SF-1 includes an activation function (AF-2) located at the C-terminus of its ligand binding domain (LBD). The precise position of the AF-2 determines the transcriptional status of a receptor. For ligand-dependent receptors, agonist binding stabilizes the AF-2 helix in a conformation where it is packed tightly against the main LBD to form a charge clamp pocket, which is permits interactions with LxxLL motifs of co-activator proteins such as the steroid receptor co-activators (Darimont, B. D., Wagner, R. L., Apriletti, J. W., Stallcup, M. R., Kushner, P. J., Baxter, J. D., Fletterick, R. J., and Yamamoto, K. R. (1998). Structure and specificity of nuclear receptor-co-activator interactions. Genes Dev 12, 3343-3356; Gampe, R. T., Jr., Montana, V. G., Lambert, M. H., Miller, A. B., Bledsoe, R. K., Milburn, M. V., Kliewer, S. A., Willson, T. M., and Xu, H. E. (2000a). Asymmetry in the PPARgamma/RXRalpha crystal structure reveals the molecular basis of heterodimerization among nuclear receptors. Mol Cell 5, 545-555; Nolte, R. T., Wisely, G. B., Westin, S., Cobb, J. E., Lambert, M. H., Kurokawa, R., Rosenfeld, M. G., Willson, T. M., Glass, C. K., and Milburn, M. V. (1998). Ligand binding and co-activator assembly of the peroxisome proliferator-activated receptor-gamma. Nature 395, 137-143; Shiau, A. K., Barstad, D., Loria, P. M., Cheng, L., Kushner, P. J., Agard, D. A., and Greene, G. L. (1998). The structural basis of estrogen receptor/co-activator recognition and the antagonism of this interaction by tamoxifen. Cell 95, 927-937). In contrast, when the receptor is bound to an antagonist, the AF-2 is removed from the active position to form a large binding pocket that interacts with the LxxxIxxxL motifs of corepressor proteins such as N-COR and SMRT (Xu, H. E., Stanley, T. B., Montana, V. G., Lambert, M. H., Shearer, B. G., Cobb, J. E., McKee, D. D., Galardi, C. M., Plunket, K. D., Nolte, R. T., et al. (2002). Structural basis for antagonist-mediated recruitment of nuclear co-repressors by PPARalpha. Nature 415, 813-817).

This general mechanism is consistent with observations from several dozen crystal structures of LBD/ligand complexes of various nuclear receptors (reviewed in Li, Y., Lambert, M. H., and Xu, H. E. (2003). Activation of nuclear receptors: a perspective from structural genomics. Structure (Camb) 11, 741-746).

SF-1 and LRH-1 share 56% amino acid sequence identity in their LBDs. Inspection of the crystal structure of the LRH-1 LBD (Sablin, E. P., Krylova, I. N., Fletterick, R. J., and Ingraham, H. A. (2003). Structural basis for ligand-independent activation of the orphan nuclear receptor LRH-1. Mol Cell 11, 1575-1585) reveals that LRH-1 assumes a similar α-helical sandwich fold seen in other nuclear receptors. The LRH-1 LBD includes a large but empty ligand binding pocket. Nevertheless, the AF-2 helix adopts an active conformation in this “apo” state. Mutations that alter the mouse LRH-1 pocket do not affect its activation function, suggesting that transcriptional activation does not require the binding of a specific ligand. However, the presence of a well-formed hydrophobic pocket raises the possibility that LRH-1 and related receptors can be regulated either positively or negatively by physiologic ligands.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present inventors report herein the 1.5 Å crystal structure of the SF-1 LBD in complex with an LxxLL motif. The structure reveals a surprisingly large ligand binding pocket (˜1600 Å3) that is filled with a phospholipid. The bound phospholipid appears to stabilize the receptor in the active conformation and can be readily exchanged with other exogenous ligands. Furthermore, the binding of co-activators to SF-1 is enhanced or inhibited by specific types of phospholipids, depending on the length of their fatty acid side chains. Directed mutations in the SF-1 pocket revealed a strong correlation of phospholipid binding and SF-1 activation. The inventors have thus discovered that SF-1 is a ligand dependent receptor, not a ligand-independent constitutively-activated receptor according to the prior art, and show for the first time an unexpected role of phospholipids in SF-1 functions.

Based on the foregoing, and the results described below, including the functional assays, the present invention provides a method to identify a phospholipid as a cognate ligand for the orphan nuclear hormone receptor SF-1.

The invention further relates to screening methods by which agonists and antagonists of nuclear receptors, particularly SF-1 can be identified, preferably by the high throughput screening (HTS) of chemical libraries to identify high affinity ligands. The present invention will lead to the discovery of non-phospholipid organic molecules that can act as agonists and antagonists of SF-1. Such agonists and antagonists can be used to treat a variety of diseases or conditions, preferably diseases of cholesterol homoeostasis, endocrine disorders, and dyslipidemias.

The identification of phospholipids as SF-1 agonists or antagonists also provide a chemical tool to probe biology and physiology of this receptor using various known methods.

### BRIEF DESCRIPTION OF THE DRAWINGS

FIG. 1. Characterization and Crystallization of the Purified SF-1 Protein

A. Binding of various LxxLL motifs to the purified SF-1 LBD as measured by AlphaScreen™ assays. The background reading of either SF-1 or the peptides alone is less than 200. The peptide sequences are listed in experimental procedures. The results are the average of three experiments (error bars=standard deviation).

B. Crystals of the SF-1/SHP ID1 complex.

FIG. 2. Overall Structure of the SF-1/SHP ID1 complex

A. Ribbon representation of the SF-1/SHP complex in two views separated by 90°. SF-1 is colored in red and the SHP ID1 motif is in yellow. The bound phospholipid ligand is shown in space-filling representation with carbon, oxygen, nitrogen, and phosphate depicted in green, red, blue and purple, respectively.

B. Sequence alignment of the mouse and human SF-1 and LRH-1 with the drosophila FTZ-F1. The secondary structural elements are annotated below the sequence alignment, and the residues that contact the phospholipid are shaded grey. The additional length of helix H2 observed in the mouse LRH-1 structure is shown in black.

FIG. 3. The Large SF-1 Ligand Binding Pocket

A. Two 90° views of the SF-1/SHP/phospholipid ternary complex with the bound phospholipid ligand (PLD) shown in space-filling representation and the SF-1 pocket shown in white surface.

B. The entrance to the SF-1 pocket (white surface) showing the exposure of the bound phospholipid (spheres) to solvent.

C. An overlap of the SF-1/SHP complex (blue) with the mouse LRH-1 LBD structure (yellow) showing the length of the SF-1 helix H2. The movements of helices H3 and H10 that contributes to the larger SF-1 pocket are also indicated.

D and E. Two close-up views of the SF-1 pocket (white surface) with the mouse LRH-1

structure (yellow). The structural changes that contribute to the larger SF-1 pocket are also indicated.

FIG. 4. Recognition of Phospholipids by SF-1

A and B. Two views of the electron density map showing the phospholipid ligand and the surrounding SF-1 residues. The chemical moieties of phospholipid are indicated. The map is calculated with 2Fo-Fc coefficients and is contoured at 1 sigma. Key residues and chemical moiety of phospholipids are indicated.

C. MS analysis of the denaturing SF-1 showing the multiple charged species of the apo-protein and the two distinct peaks at 716 Da and 690 Da.

D. A deconvoluted mass spectrum of the denatured SF-1 shows the measured average molecular weight of 29614±0.3 Da matches the expected molecular weight of the apo-SF1 at 29615.5 Da.

E. MS/MS analysis of the 690 Da peak for C32:1 phospholipid generates a major product ion at 549 Da corresponding to C32:1 diacyl-glycerol. The 141 Da difference between the two peaks is consistent with the loss of the phosphoethanolamine moiety.

F. A MS/MS analysis of the 716 Da peak for C34:2 phospholipid generates a major product ion at 575 Da corresponding to C34:2 diacyl-glycerol. The 141 Da difference between the two peaks is consistent with the loss of a phosphoethanolamine moiety.

FIG. 5. Modulation of SF-1/Coactivator Interactions by Phospholipids

Error bars in panels A, B, C and E are standard deviations of triplicate experiments.

A. The binding of the TIF2 co-activator motif to the purified SF-1 in the absence or presence of 1.25 μM of 1,2-Didodecanoyl-sn-glycero-3-phosphoethanolamine (12PE), 1,2-dimyristoyl-sn-glycero-3-phosphoethanolamine (14PE), and 1,2-dihexadecanoyl-sn-glycero-3-phosphocholine (16PC), which are phospholipids with C12, C14 and C16 fatty acid side chains, respectively. The fold of activation by phospholipids is indicated on the top of the bars.

B. The effect of 10% ethanol on SF-1 binding to the TIF2 co-activator motif in the absence or presence of 1.25 μM of phospholipids with C12-C16 fatty acids.

C. The binding of the TIF2 co-activator motif to the purified mouse LRH-1 in the absence or presence of 1.25 μM of phospholipids with C12-C18 fatty acids.

D. Dose effects of 18PCD on SF-1/TIF2 interactions in the absence of 10% ethanol.

E. Dose response curves of the SF-1/TIF2 binding to phospholipids with fatty acids with side chains of length C12 (12PE, red squares), C14 (14PE, blue triangles), C16 (16PC, purple circles), and C18 (18PCD, black squares) in the presence of 10% ethanol.

FIG. 6. Phospholipid Binding of SF-1 Is Important for the Receptor Activation

A-C. The locations of the ten mutated SF-1 pocket residues are shown with sticks in the overall SF-1 structure. Residues that affect binding of all three co-activator motifs are colored in black and residues that show partial binding are shown in blue. In panels D-F, the results are the average of triplicate experiments (error bars=standard deviation).

D. Effects of the pocket residue mutations on SF-1 binding to co-activator peptides containing the TIF2-3, SRC1-2 and SRC1-4 LxxLL motifs, respectively.

E. Effects of 12PE (1.25 μM) on restoring TIF2 binding activity of mutated SF-1 receptors.

F. Transcriptional activity of wild-type and mutated SF-1. Expression plasmids for wild type or mutant SF-1 were cotransfected with the SF-1 reporter plasmid and luciferase activity was measured and normalized to β-galactosidase (β-gal) as an internal control. Data are from triplicate assays (±SEM).

FIG. 7 is a schematic drawing of a method for high-through-put screening (HTS) of chemical libraries for compounds that bind and modulate SF-1 activity. B-LxxLL is biotinylated LxxLL peptide. SF-1-H6 is His-tagged purified SF-1 (derivatized with a His6 tag at its amino terminus). This SF-1 has a bound phospholipid ligand).

### DETAILED DESCRIPTION OF THE PREFERRED EMBODIMENTS

The preferred embodiments of the present invention may be understood more readily by reference to the following detailed description of the specific embodiments and the Examples and Sequence Listing included hereafter.

The compact disc labeled “CRF,” which is filed concurrently with this application, contains the Sequence Listing with file name “VAN67 P312 Sequence Listing.ST25.txt,” was created on Feb. 3, 2006, has a file size of 4 kilobytes, and is incorporated herein by reference. Also filed concurrently with this application are two compact discs labeled “COPY 1” and “COPY 2,” which are identical to one another and to the compact disc labeled “CRF.” The compact disc labeled “COPY 1” contains the Sequence Listing with file name “VAN67 P312 Sequence Listing.ST25.txt,” was created on Feb. 3, 2006, and has a file size of 4 kilobytes. The compact disc labeled “COPY 2” contains the Sequence Listing with file name “VAN67 P312 Sequence Listing.ST25.txt,” was created on Feb. 3, 2006, and has a file size of 4 kilobytes.

The identification of ligands for orphan nuclear receptors has revealed novel signaling pathways for several prominent classes of lipids including retinoids, fatty acids, sterols, and lipophilic xenobiotics (Chawla, A., Repa, J. J., Evans, R. M., and Mangelsdorf, D. J. (2001). Nuclear receptors and lipid physiology: opening the X-files. Science 294, 1866-1870; Kliewer, S. A., Lehmann, J. M., and Willson, T. M. (1999). Orphan nuclear receptors: shifting endocrinology into reverse. Science 284, 757-760). The present invention is based on the use of a combination of structural, biochemical, and molecular biology techniques to provide evidence that the transcriptional activity of the orphan receptor SF-1 is regulated by another important class of lipids, namely phospholipids. The interaction of SF-1 with co-activators can be either enhanced or inhibited by phospholipids depending on the length of their fatty acid tails. These findings challenge the perception from the prior art that SF-1 is constitutively active and suggest the existence of an unexpected phospholipid signaling pathway.

Structural Basis for Ligand-Dependent Activation of SF-1

A common mechanism for activation of nuclear receptors involves positioning the C-terminal AF-2 helix to form a charge clamp pocket, which permits the receptor to interact efficiently with co-activator proteins (Li et al., 2003). Nuclear receptors use diverse structural features to stabilize the AF-2 helix in the active conformation. For ligand-dependent nuclear receptors, a straightforward mechanism is the direct interaction between the AF-2 helix and the bound ligand as is observed in the structures of LBD/ligand complexes of the glucocorticoid receptor and peroxisome proliferator-activated receptors (PPARs) (Bledsoe, R. K., Montana, V. G., Stanley, T. B., Delves, C. J., Apolito, C. J., McKee, D. D., Consler, T. G., Parks, D. J., Stewart, E. L., Willson, T. M., et al. (2002). Crystal structure of the glucocorticoid receptor ligand binding domain reveals a novel mode of receptor dimerization and co-activator recognition. Cell 110, 93-105.; Xu, H. E., Lambert, M. H., Montana, V. G., Plunket, K. D., Moore, L. B., Collins, J. L., Oplinger, J. A., Kliewer, S. A., Gampe, R. T., Jr., McKee, D. D., et al. (2001). Structural determinants of ligand binding selectivity between the peroxisome proliferator-activated receptors. Proc Natl Acad Sci USA 98, 13919-13924).

The same mechanism also results in activation of constitutive androstanol receptor (CAR) by its xenobiotic ligand TCPOBOP (Suino, K., Peng, L., Reynolds, R., Li, Y., Cha, J. Y., Repa, J. J., Kliewer, S. A., and Xu, H. E. (2004). The Nuclear Xenobiotic Receptor CAR; Structural Determinants of Constitutive Activation and Heterodimerization. Mol Cell 16, 893-905).

SF-1 contains a typical C-terminal AF-2 helix in a position that allows the conserved glutamate (located at the center of the AF-2 helix) to form a charge clamp for binding of LxxLL motifs. Although SF-1 has been considered as a constitutively active member of the nuclear receptor family, according to the present invention, it is understood to comprise a large ligand binding pocket which is filled by a phospholipid ligand. The bound phospholipid stabilizes the AF-2 helix in the active conformation through several direct contacts. The bound phospholipid also makes numerous interactions with multiple structural elements of SF-1, including helices H3, H5, H6, H7, H10, and the loop preceding the AF-2 helix. Consistent with these structural data, phospholipids with C12-16 fatty acids promote the binding of co-activators to SF-1 (FIG. 5).

Conversely, the interaction of SF-1 with co-activators is reduced by either the absence of phospholipids or by phospholipids with longer fatty acids, which would be expected to stick out of the pocket and interfere with folding of the AF-2 helix into the active conformation. Thus, binding of an appropriately sized-phospholipid ligand stabilizes the overall fold of SF-1 LBD and tethers the AF-2 helix in the active conformation.

Among the ligand-dependent nuclear receptors, SF-1 is most similar to the PPARs with respect to the size of the ligand binding pocket and the mechanism of ligand-dependent receptor activation. Both SF-1 and the PPARs contain a large pocket of 1300-1600 Å3, and ligand dependent activation of both receptors is mediated through direct contacts with their AF-2 helix (Nolte et al., 1998; Xu, H. E., Lambert, M. H., Montana, V. G., Parks, D. J., Blanchard, S. G., Brown, P. J., Sternbach, D. D., Lehmann, J. M., Wisely, G. B., Willson, T. M., et al. (1999). Molecular recognition of fatty acids by peroxisome proliferator- activated receptors. Mol Cell 3, 397-403; Xu et al., 2001).

In addition, both SF-1 and the PPARs contain an opening into their pocket that may serve as a channel for ligand exchange. The most distinct structural difference between SF-1 and the PPARs is the topology of their ligand binding pocket. The PPARs have a three-arm Y-shaped pocket that is ideally suited for binding of long chain fatty acids whereas the SF-1 pocket is an extended ellipse that is well suited for binding phospholipids. In addition, the voluminous SF-1 pocket also explains the promiscuity of SF-1 binding to heterologous phospholipids with C12-C18 fatty acid side chains. Moreover, the positioning of the ethanolamine moiety outside of the SF-1 pocket suggests phospholipids with diverse head groups, including phosphatidylcholine (FIG. 5A-E) and phosphatidylinositol, may also bind to SF-1.

Besides the above structural characteristics of a ligand dependent receptor, the ligand-regulated properties of SF-1 are further supported by the inventors' biochemical binding and results of mutagenesis studies. Six mutations that were designed to fill the ligand binding pocket interfered with co-activator binding and transcriptional activation. The strong correlation between phospholipid binding in vitro and SF-1 activation in cells exhibited by these mutants indicates an important role of phospholipid binding in SF-1 activation.

In addition, three mutations (A266W, A270W, and L348W), which are located in the back of the SF-1 pocket, can be rescued by addition of smaller lipids, in agreement with that these mutants block lipid binding by filing part of pocket. Notably, similar mutations designed to fill the mLRH-1 pocket does not affect its transcriptional activity, consistent with our data that co-activator binding is not affected by the presence of phospholipids we tested. The difference of ligand dependent properties between SF-1 and mLRH-1 can be attributed to the distinct structural features of these two receptors. SF-1 contains a large ligand binding pocket that is filled with a phospholipid ligand while mLRH-1 contains a smaller but empty pocket.

Evidence of an Unexpected Phospholipid Signaling Pathway

According to the present invention, phospholipids bind to SF-1 with high affinity and modulate its activity. Importantly, a strong correlation has been discovered between phospholipid binding in vitro and SF-1 activation in cell-based assays (FIGS. 6D and 6F).

Why does SF-1 bind phospholipids? One possibility is that the phospholipid serves as a structural cofactor that allows the protein to achieve a desirable conformation. In this model, phospholipids are not hormone-like signals but instead are used as structural components because they are readily available and have the required biophysical properties. The fatty acids that bind to the HNF4 family of nuclear receptors may function in this way because once bound they do not appear to be exchangeable (Dhe-Paganon, S., Duda, K., Iwamoto, M., Chi, Y. I., and Shoelson, S. E. (2002). Crystal structure of the HNF4alpha ligand binding domain in complex with endogenous fatty acid ligand. J Biol Chem 21, 21; Wisely, G. B., Miller, A. B., Davis, R. G., Thornquest, A. D., Jr., Johnson, R., Spitzer, T., Sefler, A., Shearer, B., Moore, J. T., Willson, T. M., and Williams, S. P. (2002). Hepatocyte nuclear factor 4 is a transcription factor that constitutively binds fatty acids. Structure (Camb) 10, 1225-1234).

A second, more intriguing possibility is that SF-1 binds phospholipids as a mechanism for sampling the repertoire of fatty acid-derived membrane lipids and adjusting gene transcription accordingly. The present discovery that SF-1 can readily exchange its ligands and respond differently to distinct phospholipids are consistent with this possibility. The ratio of cholesterol to phospholipids has crucial effects on cell membrane properties including fluidity and permeability, and thus must be tightly regulated. This is accomplished in part by the coordinate regulation of fatty acid and cholesterol homeostasis by the sterol regulatory element binding protein (SREBP) and liver X receptor (LXR) families of transcription factors (Chen, G., Liang, G., Ou, J., Goldstein, J. L., and Brown, M. S. (2004). Central role for liver X receptor in insulin-mediated activation of Srebp-1c transcription and stimulation of fatty acid synthesis in liver. Proc Natl Acad Sci USA 101, 11245-11250; Repa, J. J., Liang, G., Ou, J., Bashmakov, Y., Lobaccaro, J. M., Shimomura, I., Shan, B., Brown, M. S., Goldstein, J. L., and Mangelsdorf, D. J. (2000). Regulation of mouse sterol regulatory element-binding protein-1c gene (SREBP-1c) by oxysterol receptors, LXRalpha and LXRbeta. Genes Dev 14, 2819-2830).

Because SF-1 regulates a large number of genes involved in sterol biosynthesis and homeostasis, according to the present invention, modulation of SF-1 transcriptional activity by phospholipids is provides a direct mechanism for sensing the phospholipid content of cells and transducing this information into changes in the expression of genes that would provide for maintenance of an appropriate phospholipid:sterol balance. This may be particularly important in steroidogenic tissues and cells, which are subject to large and rapid fluxes in sterol concentrations.

Since SF-1 transcriptional activity can be either enhanced or repressed depending on the specific phospholipid ligand, identification of endogenous phospholipids that bind to SF-1 will help in understanding exactly how this pathway is regulated in vivo.

Furthermore, the characterization herein of SF-1 as a ligand dependent receptor provides a conceptual framework to design synthetic SF-1 ligands with better pharmacokinetic properties than phospholipids.

The ability of the synthetic SF-1 ligands to activate or repress the receptor is particularly useful to unravel the biology mediated by SF-1, as well as in applications as pharmaceutical agents for treatment of SF-1 related diseases.

The finding that SF-1 can be regulated by ligands has important implications for other orphan nuclear receptors. SF-1-related receptors comprise an ancient subfamily within the nuclear receptor superfamily. Among the SF-1-related receptors are the Drosophila protein Ftz-F1, which plays an essential role in pattern formation during development, and the mammalian receptor LRH-1, which is required for early mammalian embryogenesis and regulates cholesterol metabolism and steroidogenesis in adults (Fayard, E., Auwerx, J., and Schoonjans, K. (2004). LRH-1: an orphan nuclear receptor involved in development, metabolism and steroidogenesis. Trends Cell Biol 14, 250-260).

According to the present invention, the ligand binding pocket may be conserved in FTZ-F1 and phospholipids also bind to the human LRH-1 by expanding its ligand binding pocket. Given the diverse biological processes affected by the SF-1 family, phospholipids may have profound physiological and developmental actions in an array of species.

The results disclosed herein establish that SF-1 is a ligand-regulated receptor and suggest an unexpected relationship between phospholipids and endocrine development and function. In addition, these findings also provide a conceptual framework for designing synthetic SF-1 ligands with better pharmacokinetic properties than phospholipids and form the basis for novel methods for screening chemical libraries for high affinity binding agonists or antagonists for SF-1 that are candidate drugs for treating a subjects in need of cholesterol homeostasis, endocrine disorders, and those with various dyslipidemias.

In addition to providing evidence that SF-1 is regulated by endogenous ligands, the present invention provides a conceptual framework for designing synthetic SF-1 ligands with better pharmacokinetic properties than phospholipids. Synthetic SF-1 ligands that either enhance or antagonize its activity will be valuable tools for dissecting SF-1 biology in addition to their utility as pharmaceutical agents for the treatment of SF-1-related diseases.

The preferred animal subject for treatment by compounds discovered using the present invention is a mammal, particularly human subjects. By the term “treating” is intended the administering to a subject of a pharmaceutical composition comprising an agonist or antagonist of SF-1, whether it is a phospholipid or an SF-binding mimic discovered using the screening methods of the invention or designed de novo using information from the invention.

The pharmaceutical compositions of the present invention comprise an SF-1 ligand that binds to SF-1 with high affinity and high specificity. As used in this application, a high affinity SF-1 ligand means that the dissociation constant between the ligand and SF-1 is less than 1.0 micromole, or means that the EC50 of the ligand affecting SF-1 activation is less than 1.0 micromole, or means that the EC50 of the ligand inhibiting SF-1 activation is less than 1.0 micromole. As used in this application, a high specificity SF-1 ligand means that the ligand binds to SF-1 with 10-fold more potency than it binds to other nuclear hormone receptors.

The pharmaceutical compositions of the present invention comprise a SF-1 ligand combined with pharmaceutically acceptable excipient or carrier, and may be administered by any means that achieve their intended purpose. Amounts and regimens for the administration of the SF-1 ligand can be determined readily by those with ordinary skill in the clinical art of treating any of the particular diseases. Preferred amounts are described below.

Administration may be by parenteral, subcutaneous (sc), intravenous (iv), intramuscular, intraperitoneal, transdermal, topical or inhalation routes. Alternatively, or concurrently, administration may be by the oral route. The dosage administered will be dependent upon the age, health, and weight of the recipient, kind of concurrent treatment, if any, frequency of treatment, and the nature of the effect desired.

Compositions within the scope of this invention include all compositions wherein the SF-1 receptor ligand is contained in an amount effective to achieve its intended purpose. While individual needs vary, determination of optimal ranges of effective amounts of each component is within the skill of the art. Typical dosages comprise 0.01 to 100 mg/kg/body wt, though more preferred dosages may be readily determined without undue experimentation.

As stated above, in addition to the pharmacologically active molecule, the pharmaceutical preparations may contain suitable pharmaceutically acceptable carriers comprising excipients and auxiliaries which facilitate processing of the active compounds into preparations which can be used pharmaceutically as is well known in the art. Suitable solutions for administration by injection or orally, may contain from about 0.01 to 99 percent, active compound(s) together with the excipient.

Having now generally described the invention, the same will be more readily understood through reference to the following examples which are provided by way of illustration, and are not intended to be limiting of the present invention, unless specified.

### EXAMPLES

### Example 1

### Experimental Procedures Used in Subsequent Examples

Protein Preparation

The mouse SF1 LBD (residues 221-462) was expressed as a 6×His fusion protein from the expression vector pET24a (Novagen). BL21 (DE3) cells transformed with this expression plasmid were grown in LB broth at 16° C. to an OD600 of ˜1.0 and induced with 0.1 mM isopropylthio-β-D-galactoside (IPTG) for 16 hours. Cells were harvested, resuspended in 400 ml extract buffer (20 mM Tris pH8.0, 150 mM NaCl, 10% glycerol) per 6 l. of cells, and passed 3 times through a French Press with pressure set at 1000 pa. The lysate was centrifuged at 20,000 rpm for 30 minutes and the supernatant was loaded onto a 50 ml Ni-NTA column (Qiagen). The column was washed with extract buffer and the protein eluted with 300 ml gradient to buffer B (10 mM Tris, PH8.0, 150 mM NaCl, 10% glycerol and 500 mM imidazole). The protein was further purified with a Q-Sepharose column (Amersham Biosciences). A typical yield of the purified protein was about 100 mg from each liter of cells. To prepare the protein-cofactor complex, we added a 2-fold excess of the SHP peptide (ASHPTILYTLLSPGP; SEQ ID NO:1) to the purified protein, followed by filter concentration to 15 mg/ml.

Crystallization and Data Collection

The SF1 crystals were grown at room temperature in hanging drops containing 1.0 μl of the above protein-peptide solutions and 1.0 μl of well buffer containing 15% PEG 3350, 100 mM KH2PO4 and 20% Glycerol. Crystals appeared within 1-2 days and continued to grow to a size up to 100 micron within a week. Before data collection, crystals were flash frozen in liquid nitrogen.

The SF1 crystals formed in the P41 212 space group, with a=73.2 Å, b=73.2 Å, c=115.7 Å, α=β=γ=90°. The 1.5 Å data set was collected with a MAR165 CCD detector at the ID line of sector-32 at the Advanced Photon Source. The observed reflections were reduced, merged and scaled with DENZO and SCALEPACK in the HKL2000 package (Otwinowski, Z., and Minor, W. (1997). Processing of x-ray diffraction data collected in oscillation mode. Methods in Enzymology 276, 307-326).

Structure Determination and Refinement

The structure of the SF-1/SHP ID1 complex was determined by molecular replacement with the AmoRe program (Navaza, J., Gover, S., and Wolf, W. (1992). AMoRe: A new package for molecular replacement. In Molecular Replacement: Proceedings of the CCP4 Study Weekend, E. J. Dodson, ed. (Daresbury, UK, SERC), pp. 87-90) using the structure of the LRH-1/SHP ID1 complex (unpublished results). A single distinct solution was obtained with a correlation coefficient of 37.3% and an R-factor of 49.4%, consistent with one complex within each asymmetry unit. The phases from the molecular replacement solution were extensively refined with solvent flattening and histogram matching using CNS, which produced a clear map for the SF-1 LBD and the SHP LxxLL motif peptide. The extra density that is compatible with a phospholipid ligand was also evident in the initial map and became even clearer through structure refinement. Manual model building was carried out with QUANTA (Accelrys, Inc), and structure refinement preceded with CNS (Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., et al. (1998). Crystallography & NMR system: A new software suite for macromolecular structure determination. Acta Crystallogr D Biol Crystallogr 54, 905-921), using the maximum likelihood target and NCS constraints. The pocket volume was calculated with Voidoo using the program default parameters and a probe with radius of 1.95 Å (leywegt, G. J., and Jones, T. A. (1994). Detection, delineation, measurement and display of cavities in macromolecular structures. Acta Cryst D, 178-185).

Binding Assays

All phospholipids were purchased from Sigma and stored according to the manufacturer's instructions. The binding of the various peptide motifs to SF-1 was determined by AlphaScreen™ assays from Perkins-Elmer as described recently for other nuclear receptors (Xu et al., 2002). Wild type SF-1 and the mutated LBDs were purified as 6×His tag fusion proteins for the assays. The experiments were conducted with approximately 20-30 nM receptor LBD and 20 nM of biotinylated TIF2 peptide or other co-activator peptides in the presence of 5 μg/ml donor and acceptor beads in a buffer containing 50 mM MOPS, 50 mM NaF, 0.05 mM CHAPS, and 0.1 mg/ml bovine serum albumin, all adjusted to pH 7.4. An AlphaScreen kit for detection of hexa-His was used in this experiment and the binding signals for the TIF2/SF-1 interaction were detected in the absence or the presence of exogenous phospholipids. EC50/IC50 values for the effects of phospholipids on TIF2 binding to SF-1 were determined from a nonlinear least square fit of the data based on an average of three repeated experiments with standard errors typically less than 10% of measurements.

The peptides with an N-terminal biotinylation are listed below.

| | | |
|---|---|---|
| TIF2-3: | QEPVSPKKKENALLRYLLDKDDTKD | (SEQ ID NO:2) |
| SRC1-2: | SPSSHSSLTERHKILHRLLQEGSP | (SEQ ID NO:3) |
| SRC1-4: | QKPTSGPQTPQAQQKSLLQQLLTE | (SEQ ID NO:4) |
| SHP-ID1: | PCQGSASHPTILYTLLSPGP | (SEQ ID NO:5) |
| SHP-ID2: | VABAPVPSILKKILLEEPNS | (SEQ ID NO:6) |
| TRAP220: | GHGEDFSKVSQNPILTSLLQITGN | (SEQ ID NO:7) |
| CBP: | SGNLVPDAASKHKQLSELLRGGSG | (SEQ ID NO:8) |
| NCOR2: | GHSFADPASNLGLEDIIRKALMGSF | (SEQ ID NO:9) |
| SMRT2: | QAVQEHASASTNMGLEAIIRKALMGKY | (SEQ ID NO:10) |

Mass Spectrometry

All analyses were performed using an electrospray quadruple time-of-flight mass spectrometer (Qtofl, Micromass, Manchester UK). The average molecular weight of the SF-1 protein was determined under denaturing conditions (50% acetonitrile, 1% acetic acid) using the standard electrospray ion source. Sample was dialyzed against 10 mM ammonium acetate pH 6.8 and further concentrated on a C4 ZipTip (Millipore). Sample was eluted from the ZipTip with 50% acetonitrile/1% acetic acid in water and flow injected at 0.5 μL/min using a Harvard Model 22 syringe pump (Harvard Apparatus). The average molecular weight was calculated using MaxEnt1 program in MassLynx v3.5 software (Micromass). Non-covalent complex analysis was performed by nanoelectrospray using Econo12 needles from New Objectives (Loo, J. A. (1997). Studying noncovalent protein complexes by electrospray ionization mass spectrometry. Mass Spectrom Rev 16, 1-23; Robinson, C. v. (2002). Spectrometry Characterization of Multiprotein Complexes. Protein-Protein Interactions, Cold Spring Harbor Laboratory Press); Rostom, A. A., and Robinson, C. V. (1999). Disassembly of intact multiprotein complexes in the gas phase. Curr Opin Struct Biol 9, 135-141). Sample was desalted by 3 buffer exchanges using microcon-10 CM concentrators (Amincon) against 10 mM ammonium acetate pH 6.8 and diluted to an approximate concentration of 15 μM. Non-covalent analysis conditions were optimized using horse heart myoglobin. Pressure in the ion source and intermediate regions were optimized using a valve connected to the rotary vacuum pump. Tandem mass spectrometric analysis (MS/MS) on apparent lipid species at 690 Da and 716 Da were obtained using the denaturing conditions as described above. Calibration of the mass spectrometer was performed using glufibrinogen peptide (Sigma) in the MS/MS mode.

Transient Transfection Assay

The mouse SF1 expression plasmid and p65-luc reporter plasmid, which contains five copies of the 21-hydroxylase-65 element upstream of the prolactin promoter (Wilson et al., 1993), were received from Dr. Keith Parker. All mutant SF1 plasmids were created using the Quick-Change site-directed mutagenesis kit (Stratagene). HepG2 human hepatoma cells were maintained in MEM containing 10% fetal bovine serum (FBS) and were transiently transfected in medium containing 5% fetal bovine serum using FuGENE6 (Roche) according to the manufacturer's protocol. Microplates (96-well) were inoculated with 3×104 cells 24 hr prior to transfection. Cells were transfected in Opti-MEM with 120 ng of reporter plasmid, 20 ng of pCMV-β-gal, and 40 ng of receptor expression vector. 36 hours after transfection, cells were harvested and luciferase and β-gal activities measured. Luciferase data were normalized to β-galactosidase as an internal control. All assays were performed in triplicate.

### Example II

Protein Characterization and Structure Determination

The mouse SF-1 LBD was expressed in bacteria and purified to homogeneity through nickel affinity and anion exchange chromatography.

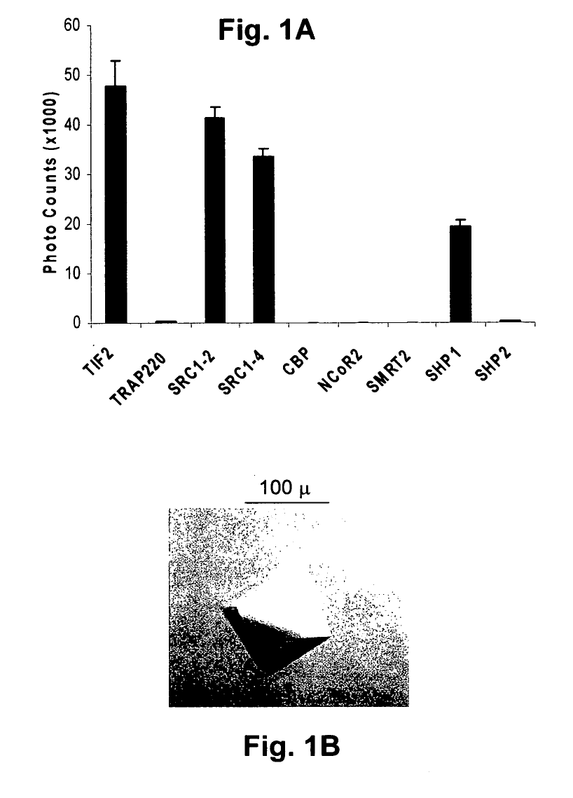

To determine the functional activity of the purified protein, AlphaScreen assays were performed to measure the interactions of the receptor with a peptide containing the third LxxLL motif from the co-activator TIF2. As shown in FIG. 1A, incubation of both SF-1 and the TIF2 peptide yielded ˜45,000 photo counts, indicating the binding of the TIF2 peptide to the receptor. The purified SF-1 is also able to interact with the second and fourth LxxLL motifs from the co-activator SRC-1 as well as the first LxxLL motif of SHP but not with the second SHP LxxLL motif or the LxxLL motifs from co-activators TRAP220 and CBP (FIG. 1A). SF-1 did not interact with peptides containing the second LxxxIxxLL (SEQ ID NO:11) co-repressor motif of SMRT or N-COR (Hu, X., and Lazar, M. A. (1999). The CoRNR motif controls the recruitment of corepressors by nuclear hormone receptors. Nature 402, 93-96; Nagy, L., Kao, H. Y., Love, J. D., Li, C., Banayo, E., Gooch, J. T., Krishna, V., Chatterjee, K., Evans, R. M., and Schwabe, J. W. (1999). Mechanism of corepressor binding and release from nuclear hormone receptors. Genes Dev 13, 3209-3216; Perissi, V., Staszewski, L. M., McInerney, E. M., Kurokawa, R., Krones, A., Rose, D. W., Lambert, M. H., Milburn, M. V., Glass, C. K., and Rosenfeld, M. G. (1999). Molecular determinants of nuclear receptor-corepressor interaction. Genes Dev 13, 3198-3208). Together these results suggest that the SF-1 protein purified from E. coli adopts an active conformation that can interact with specific co-activators but cannot interact with corepressor motifs from SMRT or N-COR.

Since inclusion of LxxLL motifs has been crucial for crystallization of a number of nuclear receptor/LBD complexes (Bledsoe et al., 2002; Gampe, R. T., Jr., Montana, V. G., Lambert, M. H., Wisely, G. B., Milburn, M. V., and Xu, H. E. (2000b). Structural basis for autorepression of retinoid X receptor by tetramer formation and the AF-2 helix. Genes Dev 14, 2229-2241; Xu et al., 2001), the SF-1 LBD complex was prepared with the peptides that interact with SF-1. Among these complexes, crystals of SF-1 bound to the first SHP LxxLL motif (ID 1) were readily obtained in the P41212 space group and diffracted to 1.5 Å (FIG. 1B). The structure was determined by the molecular replacement method using the structure of the LRH-1/SHP ID1 complex. There is only one SF-1/SHP complex in each asymmetry unit. Packing interactions between symmetry molecules in the crystal are minimal and do not constitute a functional dimer interface, in agreement with the monomeric nature of SF-1 (Wilson et al., 1993). The electron density map calculated from the molecular replacement solution revealed clear features for the binding mode of the SHP ID1 motif and the SF-1 LBD, including the entire length of helix H2 and the AF-2 helix. The data statistics and the refined structure are summarized in Table 1.

TABLE 1
Statistics of Data and Structure

| Crystal | SF1/SHP1 complex |
|---|---|
| X-ray Source | APS-32ID |
| Space Group | P4₁2₁2 |
| Resolution (Å) | 10.0-1.50 Å |
| Unique Reflections | 51,068 |
| Completeness | 100% |
| 1/σ (last shell) | 34.8 (3.0) |
| R_{sym}^{a} Mosaicity | 12.5% 0.32 |

Refinement Statistics

| | |
|---|---|
| R factor^{b} | 22.70% |
| R free | 23.92% |
| r.m.s.d. bond lengths | 0.0086 Å |
| r.m.s.d. bond angles | 1.9811° |
| total non-hydrogen atoms | 2468 |

r.m.s.d is the root mean square deviation from ideal geometry.
${}^{a} {R}_{sym} = \sum \frac{\vert Iavg - Ii \vert}{\sum Ii}$
${}^{b} {R}_{factor} = \sum \frac{\vert {F}_{P} - {F}_{Pcalc} \vert}{\sum {F}_{p}}$
where Fₚ and F_{pcalc} are observed and calculated structure factors.
R_{free} was calculated from a randomly chosen 8% of reflections excluded
from refinement and R_{factor} was calculated for the remaining 92% of
reflections.

### Example III

Structure of the SF-1 LBD/SHP Complex

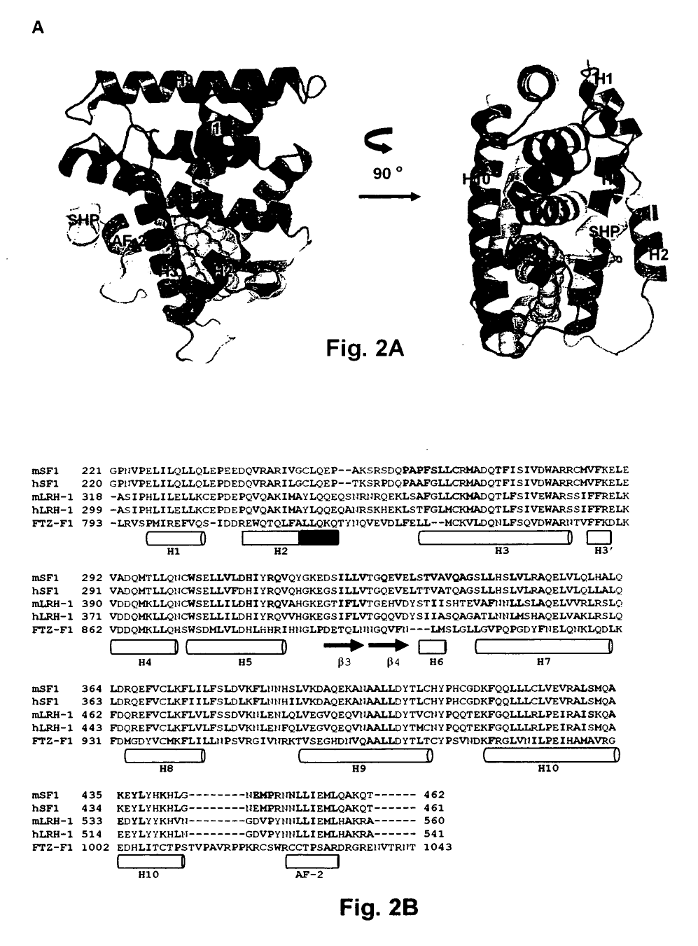

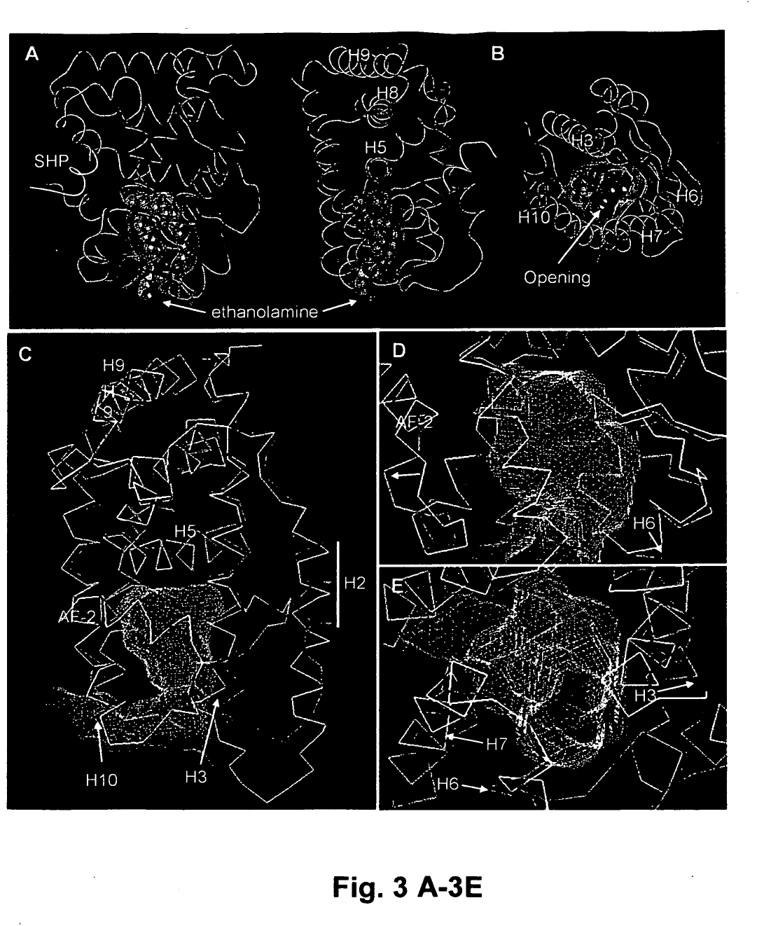

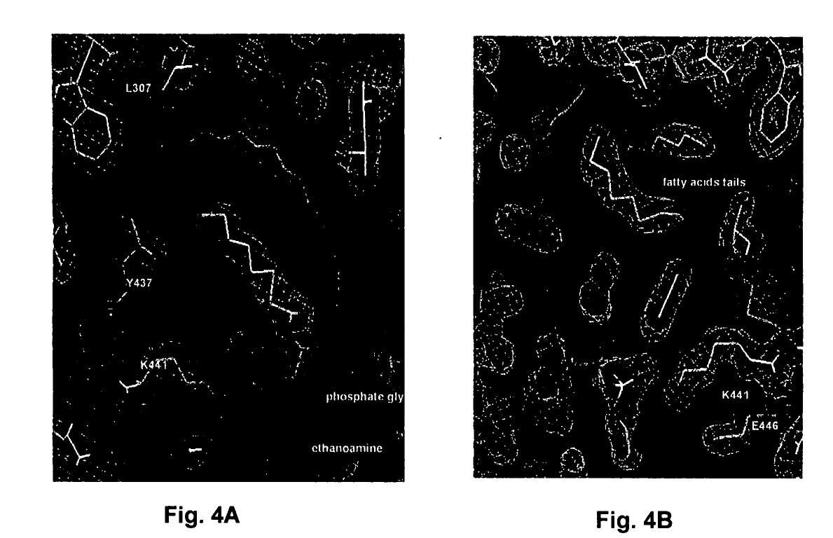

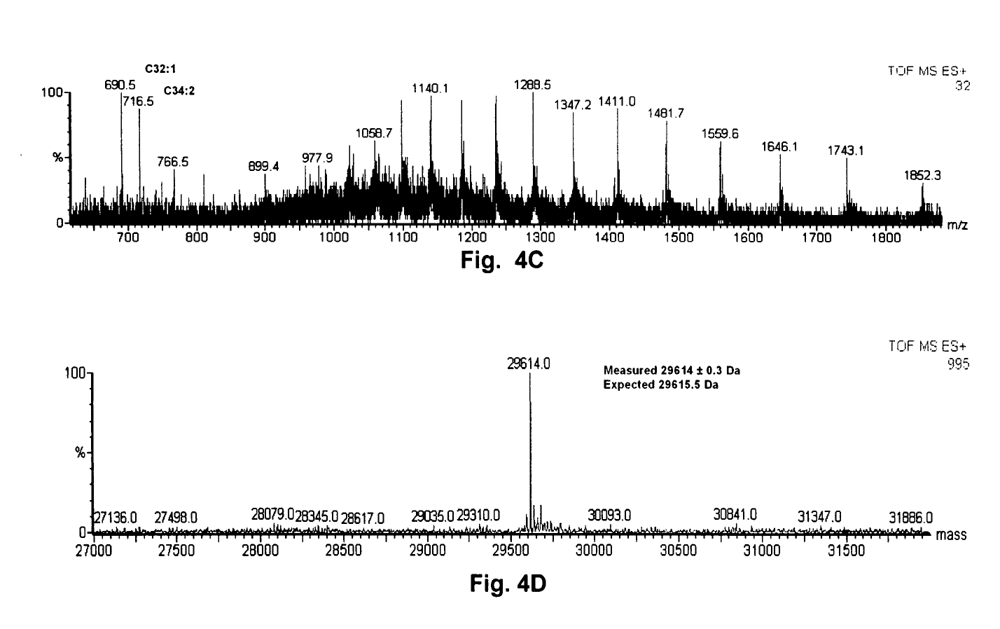

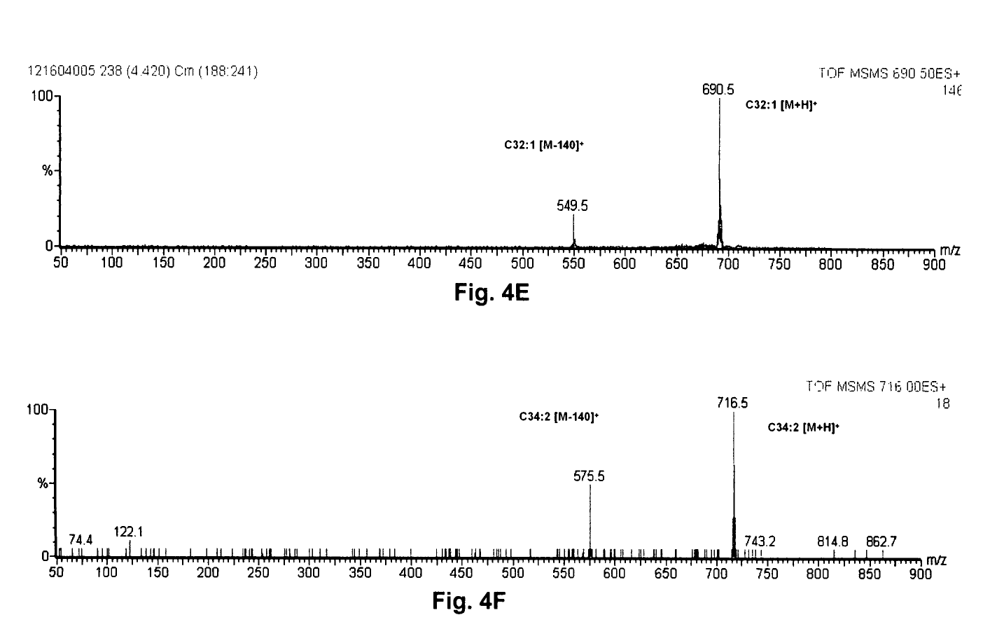

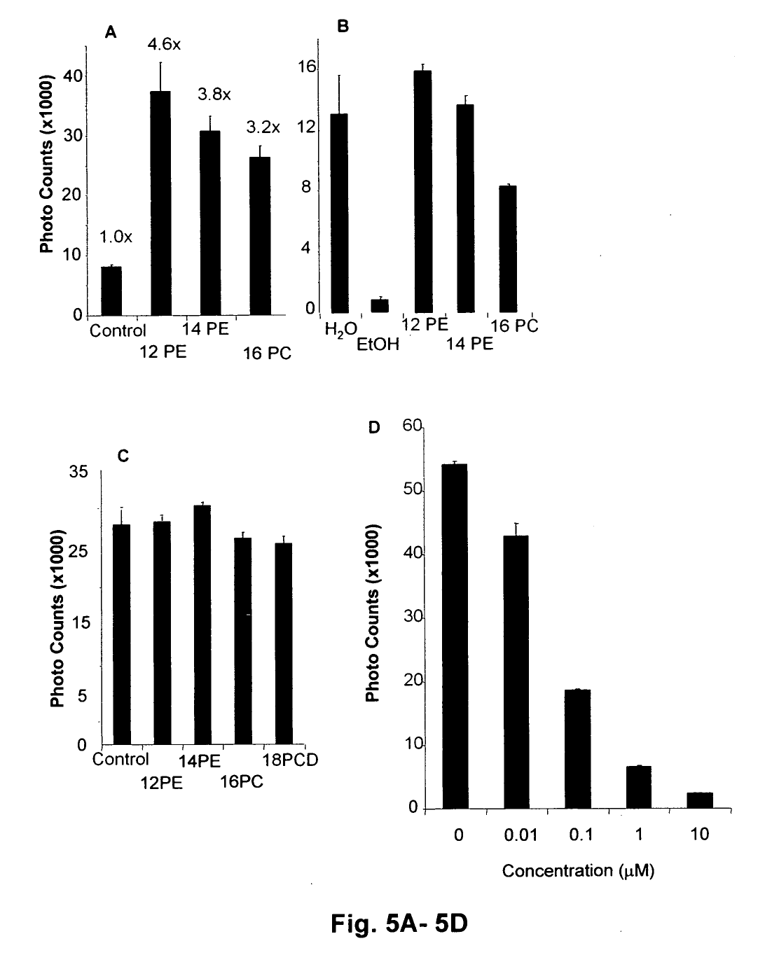

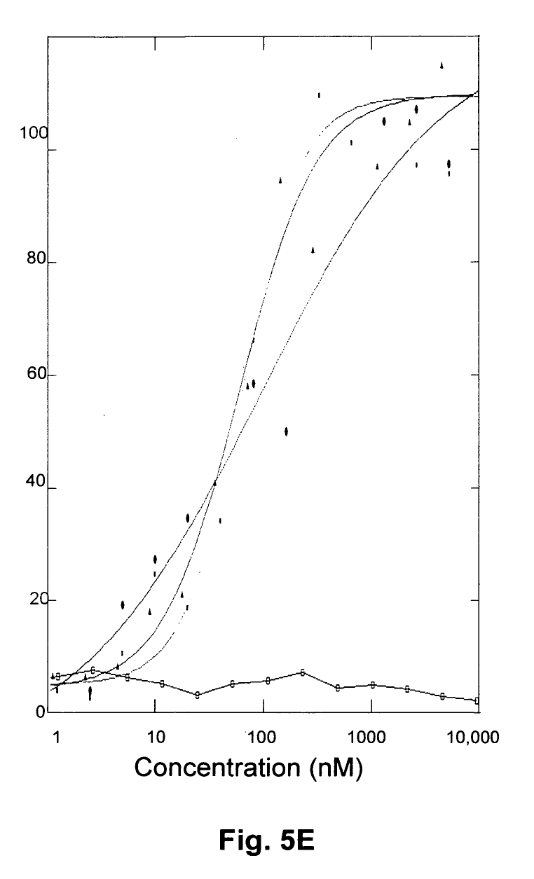

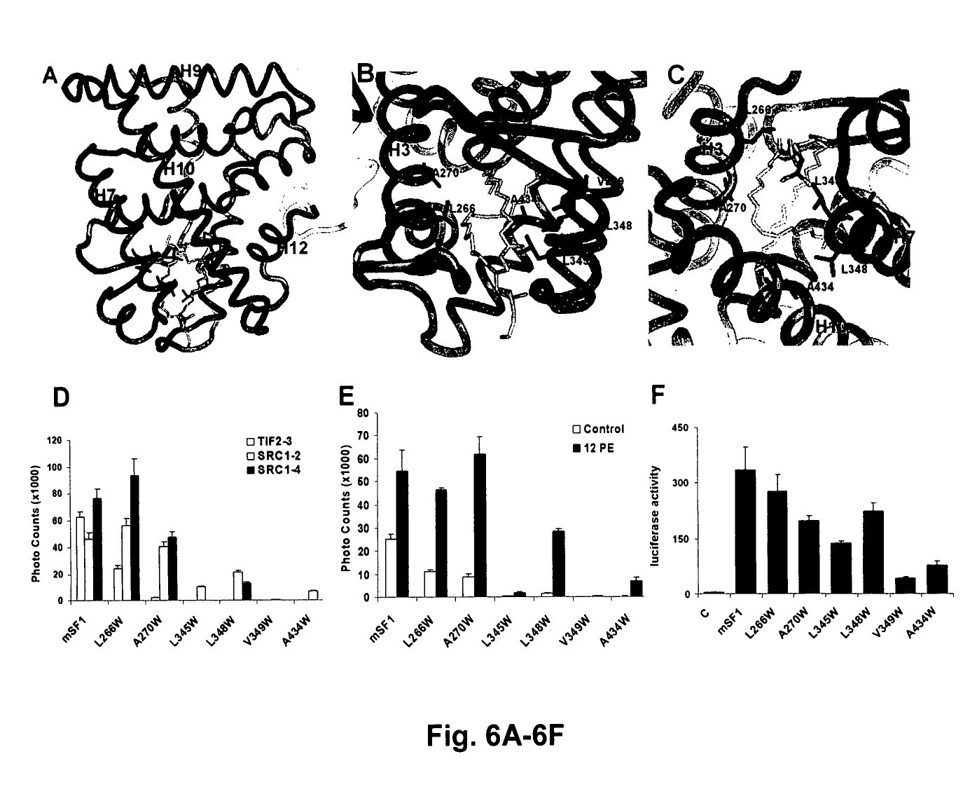

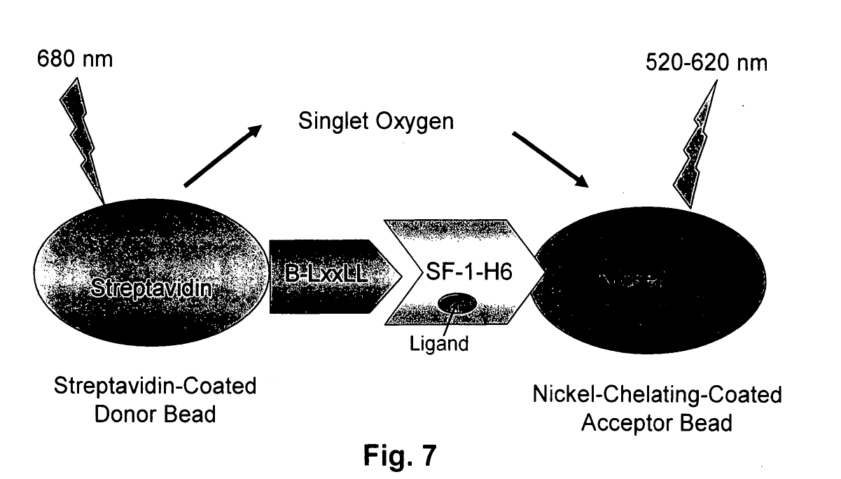

FIG. 2A shows a two-90° view of the overall arrangement of the SF-1 LBD/SHP ID1 complex. The SF-1 LBD contains 12 α-helices and two short β-strands that are folded into a typical helix sandwich. The C-terminal AF-2 is positioned in the active conformation, where it forms a charge clamp pocket to interact with the SHP ID1 motif. Similar to other LBD structures bound to co-activator LxxLL motifs, the SHP ID1 LxxLL motif adopts a two turn □ helix that directs the three hydrophobic leucine side chains into the SF-1 co-activator binding surface. The overall structure and the docking mode of the SHP ID1 motif resemble structures of co-activator LxxLL motifs bound to other nuclear receptor LBDs (Darimont et al., 1998; Gampe et al., 2000a; Nolte et al., 1998; Shiau et al., 1998).

Consistent with their sequence homology (FIG. 2B), SF-1 and LRH-1 share a very similar core LBD structure with an RMSD of 1.2 Å for the Cα atoms from helices 3-12 (FIG. 3C). Despite this similarity, there are three major differences between the structures of these two receptors. The first and most unexpected difference is the length of helix H2. In LRH-1, helix H2 is relatively long and is packed parallel to helix H3 as the fourth helical layer of the receptor. The relatively rigid nature of the LRH-1 helix H2 has been proposed as the basis for LRH-1 constitutive activity since this helix helps to stabilize the N-terminus of helix H3 and the AF-2 helix in the active conformation. However, the length of the SF-1 helix H2 is only about half that of the mouse LRH-1. As seen in FIGS. 2B and 3C, the SF-1 helix H2 lacks the C-terminal three helical turns of the LRH-1 helix H2 that pack tightly against helix H3. Thus, a key structural component of LRH-1 associated with constitutive expression is missing in the SF-1 structure, suggesting that the activation properties of SF-1 are significantly different from those of the mouse LRH-1 gene.

### Example IV

SF-1 Contains a Large Ligand Binding Pocket Filled with a Phospholipid

The second and most prominent difference between the structures of the mouse SF-1 and LRH-1 LBD is the surprisingly large ligand binding pocket of SF-1 (FIG. 3A). The accessible volume of the SF-1 pocket is 1640 Å3, which is more than twice that of the 800 Å3 mouse LRH-1 (mLRH-1) pocket. This enormous SF-1 pocket is elliptical in shape with a scaffold formed by helices H3, H5, H6, H7, H10 and the two β-strands. The C-terminal AF-2 helix and the loop preceding this helix also form one side of the pocket. Unlike the completely enclosed pocket in mLRH-1, the SF-1 pocket has a ˜100 Å2 opening at the bottom that is surrounded by the ends of helices H3, H6, H7 and H10 (FIG. 3B). This opening is directly exposed to solvent and thus may allow ligands access to the pocket. In addition, the loop preceding helix H3 is the only portion not visible in the SF-1 structure, suggesting the pocket opening is highly flexible to accommodate ligand entry or exit.

The SF-1 pocket is predominantly hydrophobic with only two small hydrophilic patches for possible polar interactions. One polar patch is composed of residues Y437, K441 and E446 from the C-terminal portion of helix H10 and Q340 from Helix H6. Together, these residues form the entrance to the pocket. The other polar patch is composed of residues H311 and R314 from the C-terminal half of helix H5 and the backbone amine of V327 from the β-turn. The structural feature comprising R314 and the backbone amine of V327 of the β-turn is conserved in RXR, RAR and TR, where it functions as an acid-binding motif to interact with the carboxylate group of retinoids and thyroid hormone (Gampe et al, 2000a; Gampe et al, 2000b; Renaud, J. P., Rochel, N., Ruff, M., Vivat, V., Chambon, P., Gronemeyer, H., and Moras, D. (1995). Crystal structure of the RAR-gamma ligand-binding domain bound to all-trans retinoic acid. Nature 378, 681-689; Wagner, R. L., Apriletti, J. W., McGrath, M. E., West, B. L., Baxter, J. D., and Fletterick, R. J. (1995). A structural role for hormone in the thyroid hormone receptor. Nature 378, 690-697). In SF-1, this acid binding motif is covered with four well structured waters, which neutralizes the charge surface and may help to stabilize the protein.

Given the relatively high homology between SF-1 and LRH-1 (FIG. 2B), it was surprising to discover that the SF-1 pocket is so much larger than the mLRH-1 pocket. Superposition of SF-1 with mLRH-1 reveals that despite the high degree of overlap in the top halves of the helices, there is significant divergence in the bottom half (FIG. 3C). Specifically, the SF-1 helix H6 and the N-terminus of helix H3 move 2.7-3.5 Å toward the right compared with mLRH-1, and the C-terminus of helix H10, the AF-2 helix, and its preceding loop move 2.0-4.3 Å toward the left (FIGS. 3D and 3E). The movement of these structural elements extends the boundaries of the SF-1 pocket to the bottom of the receptor and effectively doubles the pocket volume.

The third major difference between the SF-1 and mLRH-1 LBD structures is the presence of a phospholipid in the large SF-1 pocket, which contrasts with the empty pocket of the mouse LRH-1 LBD. This was surprising because no specific ligand was added during protein purification or crystallization. However, the high resolution of the SF-1 structure (1.5 Å) provides an exceptionally clear electron density that unambiguously reveals the binding mode of the phospholipid ligand within the SF-1 pocket. As shown in FIGS. 4A and 4B, the phospholipid includes two long fatty acids, which account for the two snake-shaped arms of density. The length of these arms suggests that the two fatty acids are both approximately 16 carbons in length. In addition, the two fatty acid arms join in a region of I-shaped density that represents a phosphate glycerol group. The ethanolamine moiety, which is attached to the phosphate group, is also clearly represented by the extra density extended beyond the phosphate group (FIG. 4A).

To characterize the bound phospholipid in the SF-1 pocket, mass spectrometry was performed with the purified SF-1 protein that was used in crystallization experiments. Nondenaturing time of flight mass spectrometry identified the expected SF-1 protein with MW of 29,614 Da and an additional peak with a heterogeneous mass distribution that corresponds to the pure protein plus extra molecular weight of 700±40 Da. In addition to the expected multiply charged ion species for SF-1 protein that correspond to the pure SF-1 (FIGS. 4C and 4D), denaturing electrospray mass spectrometry revealed two major peaks at 716 Da and 690 Da (FIG. 4C). MS/MS of these two major peaks revealed a loss of molecular weight of 141 Da, corresponding to the phosphoethanolamine moiety of a phospholipid (FIGS. 4E and 4F). MS/MS analysis also determined the identities of the fatty acid tails as C34:2 (575.5 Da) for the 716 Da peak and C32:1 (549.5Da) for the 690 Da peak.

The mass spectrometry results were completely consistent with the electron density maps showing SF-1 bound to phospholipid. However, in contrast to the multiple phospholipids identified by mass spectrometry, the 1.5 Å resolution electron density map indicates that the bound phospholipid is predominantly a C32:1 phosphatidylethanolamine with two C16 fatty acids. The dominant presence of the C32:1 phospholipid may be the result of the crystallization process, which selects conformational homogeneity of the protein during crystal packing and growth. Unsaturated fatty acids with a cis-double bond normally adopt a kink configuration at the unsaturated bond position (Xu et al., 1999). Inspection of the conformation of the bound phospholipid reveals that the monounsaturated fatty acid (C16:1) appears to be attached to the second position of the glycerol moiety with a kink roughly at positions C8-10. In addition, the phospholipid also has a chiral center at the second carbon of the glycerol moiety and it is clear that the two fatty acids in the bound phospholipid adopt a syn conformation (FIG. 4A).

The binding of the phospholipid is compatible with the chemical environment of the SF-1 pocket. The two hydrophobic fatty acid tails are completely embedded within the SF-1 hydrophobic pocket and make numerous interactions with the protein (FIGS. 4A and 4B). The glycerol-phosphate head group adopts a configuration that is parallel to helix H6 with the phosphate moiety at the pocket entry, where it makes an intricate network of hydrogen bonds with the polar and charged residues that constitute the entry site. The ethanolamine moiety is partially exposed to the solvent but forms a charged interaction with E446 from helix H10. The extensive interactions between the bound phospholipids and SF-1 suggest a high affinity interaction that accounts for the retention of the phospholipid in the pocket through multiple purification steps.

Remarkably, the bound phospholipid makes a number of direct interactions with the AF-2 helix (L453) and the loop (M447) preceding the AF-2 helix. These interactions are likely to stabilize the AF-2 helix in the active conformation and serve as a molecular basis for ligand-dependent activation of SF-1 (see below).

### Example V

Phospholipids Regulate SF-1/Coactivator Interactions

The presence in the SF-1 pocket of phospholipids that directly contact the AF-2 helix in the active conformation suggested that SF-1 is a ligand-regulated receptor, contrary to the prior art. Interactions between nuclear receptors and co-activators as measured by in vitro assays have been excellent indicators of receptor transcriptional function (Zhou, G., Cummings, R., Li, Y., Mitra, S., Wilkinson, H. A., Elbrecht, A., Hermes, J. D., Schaeffer, J. M., Smith, R. G., and Moller, D. E. (1998). Nuclear receptors have distinct affinities for co-activators: characterization by fluorescence resonance energy transfer. Mol Endocrinol 12, 1594-1604).

To determine whether SF-1 activation is ligand dependent, we monitored the interaction of SF-1 with a TIF2 co-activator LxxLL motif either in the presence or absence of phospholipids. As shown in FIG. 5A, SF-1 shows a high basal interaction with the TIF2 co-activator motif even in the absence of any exogenous ligands, consistent with the fact that a large fraction of the purified SF-1 is bound to an endogenous phospholipid. Addition of phospholipids with C12-16 fatty acids increased the SF-1I/TIF2 interaction signal by 3-5 folds, indicating that these phospholipids can function as SF-1 agonists and further promote SF-1 binding to co-activators.

The fatty acid tails of phospholipids are hydrophobic and highly soluble in hydrophobic solvents such as ethanol. Addition of 10% ethanol to the binding buffer led to >90% loss of the SF-1/TIF2 binding activity. Remarkably, SF-1/TIF2 binding activity was completely recovered by addition of phospholipids with C12-16 fatty acids (FIG. 5B), suggesting that the bound phospholipid in the SF-1 pocket is readily exchanged with exogenous ligands. The potency (EC50) of phospholipids for recovering the SF-1/TIF2 binding activity was 66 nM, 64 nM and 80-120 nM for phospholipids with C12, C14 and C16 tails, respectively (FIG. 5E). Thus, phospholipids bind to SF-1 with high affinity. In contrast, the same set of phospholipids do not affect co-activator binding by mLRH-1 (FIG. 5C), which is consistent with the finding that the mLRH-1 ligand binding pocket is only half size of the SF-1 pocket.

A comprehensive screen of other phospholipids in the SF-1/TIF2 interaction assay revealed an intriguing antagonist property for 1,2-dilinoleonyl-sn-glycerol-3-phosphocholine (18PCD), which contains two C18 fatty acid chains. While 18 PCD failed to rescue SF-1/TIF2 binding in the presence of ethanol (FIG. 5E), it disrupted the high basal interactions of SF-1 with TIF2 in a dose dependent manner (FIG. 5D). The IC50 of 18PCD for inhibiting the SF-1/TIF2 interactions was about 100-300 nM. The observation that 18PCD functions as an antagonist is consistent with the size and shape of the SF-1 pocket, which is ideally suited to accommodate a phospholipid comprised of two C12-16 fatty acids. The longer C18 fatty acids are predicted to collide with the wall of the SF-1 pocket and destabilize the canonical active conformation. Together, the present results indicate that the purified SF-1 protein is bound to a phospholipid ligand that can be readily exchanged with exogenous phospholipids. Furthermore, SF-1 interactions with co-activators are dependent on the presence of phospholipids, which can either enhance or diminish the SF-1/TIF2 co-activator interactions depending on the length of their fatty acid tails.

### Example VI

The Intact Pocket Is Important for SF-1 Activation

The SF-1 pocket comprises 51 residues that form intimate contacts with the bound phospholipid ligand. To address the role of the pocket residues in phospholipid recognition and SF-1 activation, six key residues that contact different portions of the bound phospholipid were mutated. All these mutations were designed to reduce the size of the SF-1 pocket by changing the corresponding residue to a tryptophan (W), thereby favoring the apo receptor (FIG. 6A). All mutated receptors expressed well and were readily purified for AlphaScreen assays that measure SF-1 recruitment of three different co-activator motifs that show interactions with SF-1 in FIG. 1A.

Three mutations (L345W, V349W and A434W) abolished or significantly reduced SF-1 interactions with all three co-activator motifs and the remaining three mutations (A270W, L266W, and L348W) selectively reduced SF-1 interactions with specific co-activator motifs but retained substantial binding to the other co-activator motifs (FIG. 6D).

The three mutations that retained binding to specific co-activator motifs are located either in the back of the pocket (residues A270 and L266 from H3 in FIG. 6B) or distal from the phospholipid ligand (L348, FIG. 6C). Notably, co-activator binding of these three mutants could be completely restored by addition of 12 PE, which contains short chain fatty acids and is rarely present in E. coli (FIG. 6E).

This observation is consistent with the location of these mutations, which are predicted to reduce the large size of the SF-1 pocket but still permit a smaller phospholipid to bind. In contrast, the three mutations with more pronounced effects (L345W, V349W and A434W) are positioned toward the bottom or entry of the pocket; co-activator binding to these mutants cannot be restored by 12PE, suggesting that residues that contact the phosphoglycerol group are important for phospholipid binding and co-activator recruitment.

To determine whether the in vitro co-activator binding results correlate with SF-1 transcriptional activity, cell-based assays were performed using SF-1 reporter constructs with either wild type SF-1 or the different mutants described above.

All the SF-1 pocket mutations reduced SF-1 transcriptional activity (FIG. 6F). The two mutations (V349W and A434W) that had the strongest effect in the co-activator interaction assay resulted in nearly complete loss of SF-1 transcriptional activity. The three mutants that retain substantial binding to specific co-activator motifs also retain a significant level of SF-1 transcriptional activity. These results demonstrate that residues near the bottom portion of the pocket are not only important for phospholipid binding but also for SF-1 activation in cells. The strong correlation between phospholipid binding and SF-1 transcriptional activity suggest that phospholipids serve as SF-1 ligands and regulate its transcriptional activity.

### Example VII

High-Through-Put Screening of Chemical Libraries for Compounds that Bind and Modulate SF-1 Activity

A preferred method for high-through-put screening (HTS) of chemical libraries (see FIG. 7) relies on a proximity assay such as FRET or AlphaScreen™, the latter being illustrated in the drawing. In this example SF-1/coactivator binding is measured. A donor bead is brought into proximity with an acceptor bead by interaction between the His tagged SF-1 and the biotinylated co-activator peptide. Upon excitation with a laser beam having a wavelength of 680 nm, a single donor bead emits up to 60,000 single oxygen molecules/s that activate the fluorophores in the acceptor bead to release light at a wavelength of 520-620 nm. A specific assay for SF-1 involves the following steps:

Step 1: Purify the SF-1 LBD. The purified SF-1 protein has naturally bound to it a phospholipid and thus has a high basal activity of binding co-activator (see FIGS. 1 and 5 for data).

Step 2: To screen antagonists that repress SF-1, compounds from chemical libraries are added directly to the reaction mixes to repress the high basal SF-1/coactivator binding activity (see FIG. 5E) using 18PCD. Any compound with an IC50<1 μM is considered an effective and high affinity antagonist.

To screen agonists that activate SF-1, the purified SF-1 is first incubated with ethanol or another organic solvent to remove the bound phospholipid from the SF-1 protein. Since SF-1/coactivator binding is ligand dependent, removal of phospholipid from SF-1 will reduce its co-activator binding activity by >90% (FIG. 5B). Compounds from chemical libraries are added to screen the ability to recover SF-1 co-activator binding. Any compound with EC50<1 μM is considered an effective and high affinity agonist.

The specificity of the ligand identified in Step 2 is determined by screening the same compound against other related nuclear receptors either in binding or activation assays. Any compound that binds to SF-1 with 10 fold more potency than to other nuclear receptors is considered a SF-1 ligand with high specificity.

It will be understood by those who practice the invention and those of ordinary skill in the art that various modifications and improvements may be made to the invention without departing from the spirit of the disclosed concept. The scope of protection afforded is to be determined by the claims and the breadth of interpretation allowed by the law.

What is claimed is:

1. A method for screening for a steroidogenic factor-1 receptor ligand comprising: isolating a steroidogenic factor-1 receptor ligand having a high specificity for steroidogenic factor-1 receptor.

2. A method for screening for a steroidogenic factor-1 receptor ligand comprising: isolating a steroidogenic factor-1 receptor ligand having a high binding affinity for steroidogenic factor-1 receptor.

3. The method of claim 1 wherein the isolated ligand is a steroidogenic factor-1 receptor agonist.

4. The method of claim 2 wherein the isolated ligand is a steroidogenic factor-1 receptor agonist.

5. The method of claim 1 wherein the isolated ligand is a steroidogenic factor-1 receptor antagonist.

6. The method of claim 2 wherein the isolated ligand is a steroidogenic factor-1 receptor antagonist.

7. A method for designing a steroidogenic factor-1 receptor ligand comprising: isolating a steroidogenic factor-1 receptor ligand having a high specificity for steroidogenic factor-1 receptor.

8. A method for designing a steroidogenic factor-1 receptor ligand comprising: isolating a steroidogenic factor-1 receptor ligand having a high binding affinity for steroidogenic factor-1 receptor.

9. The method of claim 7 wherein the isolated ligand is a steroidogenic factor-1 receptor agonist.

10. The method of claim 8 wherein the isolated ligand is a steroidogenic factor-1 receptor agonist.

11. The method of claim 7 wherein the isolated ligand is a steroidogenic factor-1 receptor antagonist.

12. The method of claim 8 wherein the isolated ligand is a steroidogenic factor-1 receptor antagonist.

13. A pharmaceutical composition comprising: a synthetic steroidogenic factor-1 receptor ligand having high specificity for steroidogenic factor-1 receptor.

14. A pharmaceutical composition comprising: a synthetic steroidogenic factor-1 receptor ligand having high binding affinity for steroidogenic factor-1 receptor.

15. The pharmaceutical composition of claim 13 wherein the ligand is a steroidogenic factor-1 receptor antagonist.

16. The pharmaceutical composition of claim 14 wherein the ligand is a steroidogenic factor-1 receptor antagonist.

17. The pharmaceutical composition of claim 15 wherein the ligand is a phospholipid.

18. The pharmaceutical composition of claim 16 wherein the ligand is a phospholipid.

19. The pharmaceutical composition of claim 13 wherein the ligand is a steroidogenic factor-1 receptor agonist.

20. The pharmaceutical composition of claim 14 wherein the ligand is a steroidogenic factor-1 receptor agonist.

21. The pharmaceutical composition of claim 19 wherein the ligand is a phospholipid.

22. The pharmaceutical composition of claim 20 wherein the ligand is a phospholipid.

23. A method for treating a steroidogenic factor-1 receptor-related disease comprising: administering to a subject a pharmaceutical composition having a high specificity for steroidogenic factor-1 receptor.

24. A method for treating a steroidogenic factor-1 receptor-related disease comprising: administering to a subject a pharmaceutical composition having a high binding affinity for steroidogenic factor-1 receptor.

25. The method of claim 23 wherein the pharmaceutical composition is a steroid hormone.

26. The method of claim 23 wherein the pharmaceutical composition is a phospholipid.

27. The method of claim 23 wherein the pharmaceutical composition is a steroidogenic factor-1 receptor binding mimic.

28. The method of claim 23 wherein the pharmaceutical composition is a steroidogenic factor-1 receptor agonist.

29. The method of claim 23 wherein the pharmaceutical composition is a steroidogenic factor-1 receptor antagonist.

30. The method of claim 23 wherein the steroidogenic factor-1 receptor-related disease is a disease of dyslipidemia, an endocrine disorder, or cholesterol homeostasis.

31. The method of claim 24 wherein the pharmaceutical composition is a steroid hormone.

32. The method of claim 24 wherein the pharmaceutical composition is a phospholipid.

33. The method of claim 24 wherein the pharmaceutical composition is a steroidogenic factor-1 receptor binding mimic.

34. The method of claim 24 wherein the pharmaceutical composition is a steroidogenic factor-1 receptor agonist.

35. The method of claim 24 wherein the pharmaceutical composition is a steroidogenic factor-1 receptor antagonist.

36. The method of claim 24 wherein the steroidogenic factor-1 receptor-related disease is a disease of dyslipidemia, an endocrine disorder, or cholesterol homeostasis.
